(19) United States
(12) Patent Application Publication
(10) Pub. No.: US 2025/0161053 A1
(43) Pub. Date: May 22, 2025

(54) JOINT REPLACEMENT SYSTEM ENABLING JOINT MOVEMENT

(71) Applicant: John G. Rupolo, Floral Park, NY (US)

(72) Inventor: John G. Rupolo, Floral Park, NY (US)

(21) Appl. No.: 18/921,598

(22) Filed: Oct. 21, 2024

Related U.S. Application Data
(63) Continuation-in-part of application No. 18/205,649, filed on Jun. 5, 2023.

(51) Int. Cl. A61F 2/30; A61F 2/38; A61L 27/16; A61L 27/32; A61L 27/36; A61L 27/56

(52) U.S. Cl. CPC A61F 2/30771; A61F 2/38; A61L 27/16; A61L 27/32; A61L 27/3612; A61L 27/56; A61F 2002/30581; A61F 2002/30675; A61F 2002/30691; A61F 2002/3092; A61L 2430/24

(57) ABSTRACT

A joint replacement system includes an insert configured to be disposed on an articular surface of a limb joint, a biocompatible fluid including a synthetic synovial fluid, a porous condyle configured to contain the biocompatible fluid and to be disposed on an opposing surface of the limb joint, and a sensor disposed along a top surface of the insert.

### CROSS REFERENCE TO RELATED APPLICATIONS

The present application is a Continuation-in-Part application of U.S. Non-Provisional patent application Ser. No. 18/205,649 filed on Jun. 5, 2023, the entire contents of which are hereby incorporated herein by reference.

### TECHNICAL FIELD

The present disclosure relates generally to the field of joint replacement systems. More specifically, an aspect of the present disclosure provides a joint replacement system for a knee configured to enable the efficient movement of the knee joint.

### BACKGROUND

Efficient joint movement and mobility aid in lowering physical injury in that in the event of an elevated range of joint movement, joint strength is greatly reduced, thus increasing the risk of injury. While a majority of lower lumbar pain may be attributed to muscle related issues, a large portion of patients who experience lower lumbar pain suffer from various joint problems that, in turn, lead to compromised lower lumbar segments. Certain joints, such as the knee, play a substantial role in determining how an individual's back functions and feels. Over time, wear and tear of the joints associated with age and movement may lead to the development of substantial and, in some cases, chronic lower lumbar pain that can make engaging in day-to-day activities painful and burdensome.

Accordingly, there is interest in joint replacement systems that mitigate the degradation of joints over time, in turn reducing the likelihood and severity of lower lumbar pain and promoting longevity of healthy joint movement.

### SUMMARY

An aspect of the present disclosure provides a joint replacement system. The joint replacement system includes an insert configured to be disposed on an articular surface of a limb joint, the insert fabricated at least in part from polytetrafluoroethylene; a biocompatible fluid including a synthetic synovial fluid; a porous condyle configured to contain the biocompatible fluid and to be disposed on an opposing surface of the limb joint, the porous condyle fabricated at least in part from a rubberized biocompatible material, and a sensor disposed along a first surface of the insert. The porous condyle includes an exterior surface defining a recess configured to separate a first curvilinear portion of the porous condyle from a second curvilinear portion of the porous condyle, the recess disposed between the first and second curvilinear portions of the porous condyle; an interior surface defining a cavity for containing the biocompatible fluid and a diffusible sac containing an additional volume of biocompatible fluid; a channel disposed along the exterior surface, the channel configured to drain excess biocompatible fluid within the porous condyle; and a connector member disposed within the cavity between the first and second curvilinear portions of the porous condyle, the connector member configured to enable fluid communication within the cavity between the first and second curvilinear portions of the porous condyle. The porous condyle and the insert are aligned to enable articulation of the limb joint. A first surface of the insert is continuous and configured to contact the porous condyle and a second opposing surface of the insert is configured to be disposed on the articular surface of the limb joint. The insert is secured on the articular surface of the limb joint by a biocompatible adhesive including at least one of an ultraviolet (UV) curable adhesive or a biocompatible adhesive spray.

In an aspect of the present disclosure, the limb joint may be a knee joint.

In another aspect of the present disclosure, the condyle may be configured to selectively receive an amount of the biocompatible fluid therein.

In yet another aspect of the present disclosure, the biocompatible fluid may further include at least one of glucosamine and a biomimetic synovial fluid.

In a further aspect of the present disclosure, the system may further include a sponge disposed along the opposing surface of the limb joint upon which the porous condyle rests.

In yet a further aspect of the present disclosure, the diffusible sac may be configured to selectively release an amount of the biocompatible fluid when a decrease of an existing volume of biocompatible fluid within the cavity of the porous condyle is detected.

In an aspect of the present disclosure, the system may further include a plurality of channels.

In another aspect of the present disclosure, the channel may be configured to selectively lubricate the porous condyle.

In yet another aspect of the present disclosure, the insert and the porous condyle may be configured to be in a contact relationship with one another when the limb joint is in both a flexed and extended position.

In a further aspect of the present disclosure, during articulation of the limb joint, a surface of the porous condyle may be configured to distribute a pressure applied thereon across a surface of the insert, the surface of the insert configured to absorb the applied pressure.

In yet a further aspect of the present disclosure, the sensor may be configured to detect pressure application across a surface of the insert in response to articulation of the limb joint.

In an aspect of the present disclosure, the sensor may be configured to detect degradation of the porous condyle in response to articulation of the limb joint.

An aspect of the present disclosure provides a joint replacement system. The joint replacement systems includes an insert configured to be disposed on an articular surface of a limb joint, the insert fabricated at least in part from polytetrafluoroethylene; a biocompatible fluid including a synthetic synovial fluid; and a porous condyle configured for being disposed on an opposing surface of a limb joint. The porous condyle includes a first exterior surface made at least in part from a rubberized biocompatible material defining a recess configured to separate a first curvilinear portion of the porous condyle from a second curvilinear portion of the porous condyle, the recess disposed between the first and second curvilinear portions of the porous condyle; a second exterior surface encasing the first exterior surface, wherein the second exterior surface is coated in a biocompatible coating including calcium phosphate; an interior surface defining a cavity for containing a biocompatible fluid including a synthetic synovial fluid; a diffusible sac within the cavity, wherein the diffusible sac is configured to contain an additional volume of biocompatible fluid; a channel disposed along the second exterior surface, wherein the channel is configured for drainage of excess biocompatible fluid within the porous condyle; and a connector member disposed within the cavity between the first and second curvilinear portions of the porous condyle, the connector member configured to enable fluid communication within the cavity between the first and second curvilinear portions of the porous condyle.

In an aspect of the present disclosure, the limb joint may be a knee joint.

In another aspect of the present disclosure, the biocompatible fluid may further include glucosamine and/or a biomimetic synovial fluid.

In yet another aspect of the present disclosure, the joint replacement system may further include a sponge disposed along the opposing surface of the limb joint upon which the porous condyle rests.

In a further aspect of the present disclosure, the channel may be further configured to selectively lubricate the porous condyle.

In yet a further aspect of the present disclosure, the joint replacement system may further include a sensor disposed along a first surface of the insert.

In another aspect of the present disclosure, the sensor may be configured to detect pressure application across a surface of the insert in response to articulation of the limb joint and/or degradation of the porous condyle in response to articulation of the limb joint.

An aspect of the present disclosure provides a joint replacement system. The joint replacement systems includes an insert configured to be disposed on an articular surface of a limb joint, the insert fabricated at least in part from polytetrafluoroethylene; a biocompatible fluid including a synthetic synovial fluid; a porous condyle configured to contain the biocompatible fluid and disposed on an opposing surface of the limb joint, the porous condyle fabricated at least in part from a rubberized biocompatible material, and a sensor disposed along a first surface of the insert. The porous condyle including a first exterior surface made at least in part from a rubberized biocompatible material defining a recess configured to separate a first curvilinear portion of the porous condyle from a second curvilinear portion of the porous condyle, the recess disposed between the first and second curvilinear portions of the porous condyle; a second exterior surface encasing the first exterior surface, wherein the second exterior surface is coated in a biocompatible coating including calcium phosphate; an interior surface defining a cavity for containing a biocompatible fluid including a synthetic synovial fluid; a diffusible sac within the cavity, wherein the diffusible sac is configured to contain an additional volume of biocompatible fluid; a channel disposed along the second exterior surface, wherein the channel is configured for drainage of excess biocompatible fluid within the porous condyle; and a connector member disposed within the cavity between the first and second curvilinear portions of the porous condyle, the connector member configured to enable fluid communication within the cavity between the first and second curvilinear portions of the porous condyle. The porous condyle and the insert are aligned to enable articulation of the limb joint. A first surface of the insert is continuous and configured to contact the porous condyle and a second opposing surface of the insert is configured to be disposed on the articular surface of the limb joint. The insert is secured on the articular surface of the limb joint by a biocompatible adhesive including at least one of an ultraviolet (UV) curable adhesive or a biocompatible adhesive spray.

### BRIEF DESCRIPTION OF THE DRAWINGS

A better understanding of the features and advantages of the present disclosure will be obtained by reference to the following detailed description that sets forth illustrative aspects, in which the principles of the present disclosure are utilized, and the accompanying drawings of which:

FIG. 1 is a diagram of a condyle configured for use with a joint replacement system, in accordance with aspects of the disclosure;

FIG. 2 is a cross-sectional view of the condyle of FIG. 1, in accordance with aspects of the disclosure;

FIG. 3 is a diagram of a medial to lateral flexion view of a joint replacement system, in accordance with aspects of the disclosure;

FIG. 4 is a diagram of a lateral to medial non-flexion view of the joint replacement system of FIG. 3;

FIG. 5 is a diagram of a posterior non-flexion view of the joint replacement system of FIG. 3; and

FIG. 6 is a diagram of an anterior flexion view of a condyle configured for use with the joint replacement system of FIG. 3.

### DETAILED DESCRIPTION

The present disclosure relates generally to the field of joint replacement systems. More specifically, an aspect of the present disclosure provides a joint replacement system that enables the efficient movement of a limb joint, such as a knee. The disclosed joint replacement system mitigates the degradation of joints over time, in turn minimizing the likelihood and severity of lower lumbar pain and promoting longevity of healthy joint movement. The disclosed joint replacement system weighs less than metal reinforcements of the joint, providing the benefit, over metal reinforcement, of creating a symmetric weight distribution and not causing a transfer of weight to the opposite side. This provides the additional benefit of minimizing stress and improving the range of motion. Unlike metal reinforcement of the joint, the disclosed joint replacement system will not transfer weight to the opposite limb and will not cause issues with the lower back.

Aspects of the present disclosure are described in detail with reference to the drawings wherein like reference numerals identify similar or identical elements.

Although the present disclosure will be described in terms of specific aspects and examples, it will be readily apparent to those skilled in this art that various modifications, rearrangements, and substitutions may be made without departing from the spirit of the present disclosure.

For purposes of promoting an understanding of the principles of the present disclosure, reference will now be made to exemplary aspects illustrated in the drawings, and specific language will be used to describe the same. It will nevertheless be understood that no limitation of the scope of the present disclosure is thereby intended. Any alterations and further modifications of the features illustrated herein, and any additional applications of the principles of the present disclosure as illustrated herein, which would occur to one skilled in the relevant art and having possession of this disclosure, are to be considered within the scope of the present disclosure.

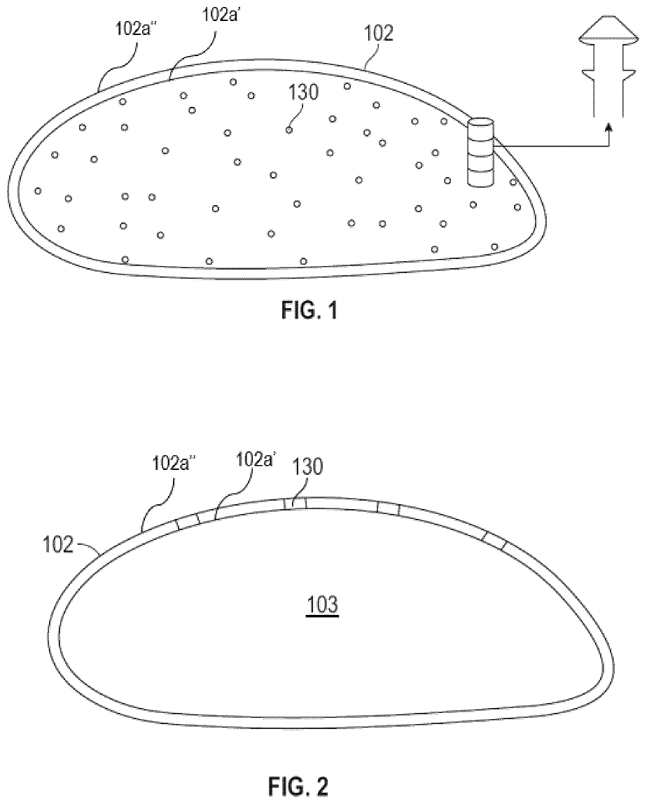

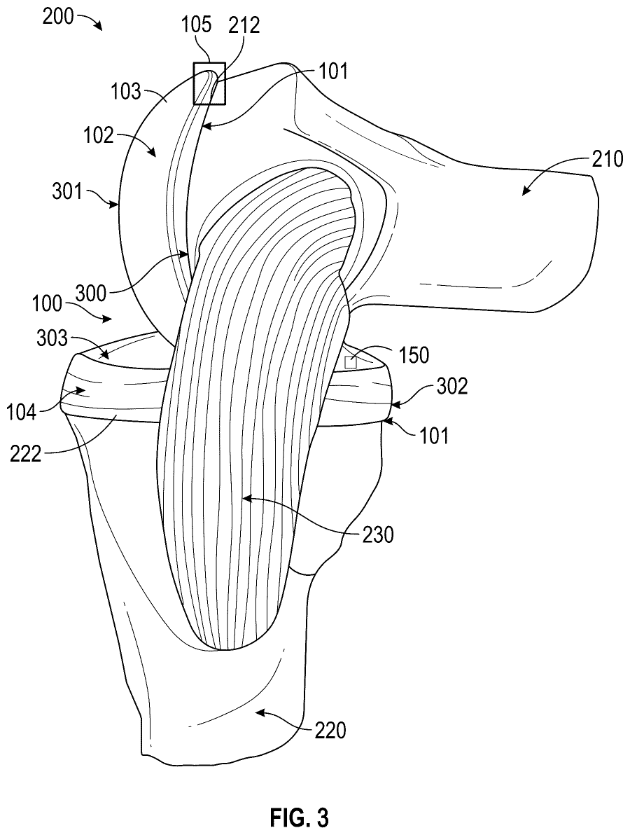

Referring to FIGS. 1-3, a porous condyle 102 (FIGS. 1-2) and a medial to lateral flexion view of a joint replacement system 100 (FIG. 3) are shown. Porous condyle 102 may be fabricated from any biocompatible material capable of being formed into a shape of a naturally occurring synovial condyle and/or containing a fluid under the forces experienced during normal physical activity of the joint into which the present system is employed. In aspects, porous condyle 102 is fabricated from a material that mimics the properties of a patient's naturally occurring synovial condyle, such as flexibility, strength and durability. For example, porous condyle 102 may be fabricated from rubberized material(s), such as silicone rubber. Namely, porous condyle 102 is made from a porous biocompatible material which may include cellulose microfibers and/or gelatin. The inclusion of a porous biocompatible material provides a benefit over traditional condyles by allowing cells and/or macromolecules to move through the porous condyle 102, heightening longevity of the porous condyle, and enabling better biological fixation. In various embodiments, a plurality of pores 130 of the porous biocompatible material may vary in dimension and/or may range from a plurality of microscopic pores to a plurality of larger porous structures. The use of porous biocompatible material provides a benefit over traditional condyles of promoting healthy bone in-growth through the porous condyle's 102 open porous geometry, thereby leading to faster osseointegration and/or healing. Porous condyle 102 may be double layered in structure, having a first exterior surface 102a′ and a second exterior surface 102a″ encasing the first exterior surface. Both the first exterior surface 102a′ and the second exterior surface 102a″ may be made from a porous biocompatible material. The second exterior surface 102a″ of the porous condyle 102 provides the added benefit of serving as a shock absorbent layer such that efficient load distribution is achieved and the degradation of the first exterior surface is minimized, thereby promoting longevity of the porous condyle 102 and minimizing the need for invasive maintenance procedures overtime. In aspects, the second exterior surface 102a″ may be coated in a biocompatible coating including calcium phosphate, collagen, gelatin, and/or cellulose. The use of this biocompatible coating aids in promoting osteointegration and, ultimately, preventing infection. In aspects, the second exterior surface may be a textured surface and/or a smooth surface.

Porous condyle 102 is configured to hold biocompatible fluid 103. Generally, biocompatible fluid 103 is fabricated from a fluid that mimics natural synovial fluid. For example, biocompatible fluid 103 may be fabricated from synthetic synovial fluids such as hyaluronan, glucosamine, and/or biomimetic synovial fluids. A second surface 300 of porous condyle 102 may be secured to an opposing surface 212 of a limb joint 200 through the use of a biocompatible adhesive 101. Suitable biocompatible adhesives include, but are not limited to cyanoacrylates and UV curable adhesives. In aspects, the biocompatible adhesive may be a liquid, gel or spray, or in the form of a biocompatible adhesive film. In various embodiments, a sponge may be first placed on the opposing surface 212 of the limb joint 200 prior to adhesion of the porous condyle 102 such that the porous condyle 102 does not sit directly on the limb joint 200. This provides a benefit over traditional joint replacement systems by promoting longevity of the porous condyle and assisting in load distribution for the joint replacement system. The sponge may be configured as a special type of absorbent material used to manage fluids, such as gauze, foam, and/or Gelfoam. Porous condyle 102 replaces and/or performs the function of a patient's synovial membrane, which may be damaged by physical trauma and/or disease or simply worn out. Porous condyle 102 may be filled with a fluid that mimics the function of the synovial fluid. Porous condyle 102, once filled with the fluid, serves as a cushion upon the end portion of bones and, ultimately, significantly reduces the amount of friction created when a joint is articulated during movement. Porous condyle 102 may be periodically refilled with biocompatible fluid 103 as needed through an injection port 105 disposed along an exterior surface of a first surface 301 of porous condyle 102. The injection port 105 may contain various configurations such as a single injection port, dual injection port, and/or a port connected to a reservoir with biocompatible fluid. In various embodiments a channel, or a plurality thereof, may be disposed along the second exterior surface 102a″ of the porous condyle 102. The channels provide a benefit of traditional condyles by enabling refilling of the biocompatible fluid 103 drainage of excess biocompatible fluid 103 within the porous condyle 102, and/or excretion of lubricant to the second exterior surface 102a″ of the porous condyle 102 for efficient articulation. The channel also provides the added benefit of refilling, draining, and/or lubricating the porous condyle 102 in a minimally invasive manner through the use of needles and injections rather than invasive procedures, which may lead to potential scarring and infection. The channel may include various configurations, such as channels interconnecting with pores, longitudinal channels running parallel to the length of the condyle to promote bone and/or tissue growth, radial channels, and/or other configurations configured to promote healthy joint articulation.

In an effort to promote minimally invasive procedures and reduce the need for operations that may lead to infections and discomfort, a diffusible sac may be disposed within the porous condyle 102. The diffusible sac may be configured to contain an additional volume of biocompatible fluid 103 such that when a decrease of an existing volume of biocompatible fluid 103 is detected within the porous condyle 102, the diffusible sac may selectively release an amount of biocompatible fluid 103 to replenish the porous condyle 102 and retain a preferred volume.

Once filled with biocompatible fluid 103, porous condyle 102 may possess a self-sealing quality to restore the porous condyle 102 to a pre-injected form. Porous condyle 102 is disposed upon opposing surface 212 of limb joint 200 to provide cushioning when pressure is placed upon a first surface 303 of an insert 104 and, ultimately, to promote efficient joint movement. The present joint replacement system 100 (FIG. 3) provides the benefit over traditional joint replacement systems of delaying gradual wear and tear of the limb joint 200 and/or significantly reducing the possibility and severity of lower lumbar pain development. In cases of patients who suffer from bone disease (e.g., osteoporosis), an additional abutment and/or bracing may be including when the limb joint 200 begins to shrink as a result of the disease, e.g., causing insert 104 to potentially come loose and become uncomfortable.

The joint replacement system 100 generally includes the insert 104 configured to be disposed on an articular surface 222 of a limb joint 200 (e.g., a knee) and porous condyle 102 configured to contain biocompatible fluid 103. Insert 104 includes the first surface 303 and a second surface 302. First surface 303 is configured to contact porous condyle 102 when the joint replacement system 100 is installed in the limb joint 200, and second surface 302 is configured to be disposed on the articular surface 222 of limb joint 200. Porous condyle 102 includes a first surface 301 and a second surface 300. Second surface 300 of porous condyle 102 is configured to be disposed on an opposing surface 212 of limb joint 200, and first surface 301 of porous condyle 102 is configured to contact first surface 303 of insert 104 when the joint replacement system 100 is installed in limb joint 200 such that insert 104 and porous condyle 102 are aligned to enable articulation of limb joint 200.

Insert 104 serves as an artificial replacement of a patient's articular cartilage, which may be damaged by physical trauma and/or disease or simply worn out. Insert 104 may be formed to match the shape of the natural articular cartilage that it serves to replace. In aspects, insert 104 may be fabricated from a lubricative biocompatible material having sufficient strength to withstand the stresses present in limb joint 200. Polytetrafluoroethylene, sometimes marketed under the trademark Teflon™, is an example of a resilient material that possesses a small likelihood of deformity and offers effective lubricative qualities.

Second surface 302 of insert 104 may be secured to articular surface 222 of limb joint 200 using a biocompatible adhesive 101 such as a cyanoacrylate and/or a UV curable adhesive. In aspects, the biocompatible adhesive may be a liquid, gel or spray, and/or in the form of a biocompatible adhesive film. Alternatively, the usage of surgical screws or other fasteners may be used to secure the second surface 302 of insert 104 to the articular surface 222 of limb joint 200. First surface 303 of insert 104 receives the shock and pressure associated with the articulating limb joint, as transferred by first surface 301 of porous condyle 102 which is positioned on opposing surface 212 of limb joint 200. First surface 303 of insert 104 serves as a continuous plateau upon which first surface 301 of porous condyle 102 may efficiently move to provide a smooth, lubricated surface for articulation and/or to facilitate the transmission of loads with a low frictional coefficient. As used here, “continuous” means a flat, smooth, and/or unbroken, which may exclude interruptions, indentations, holes, and/or discontinuities therein.

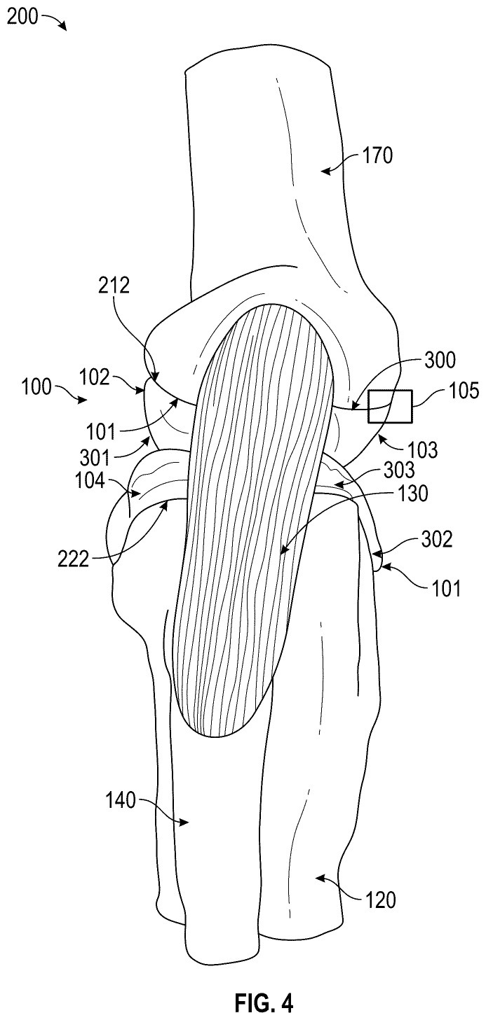

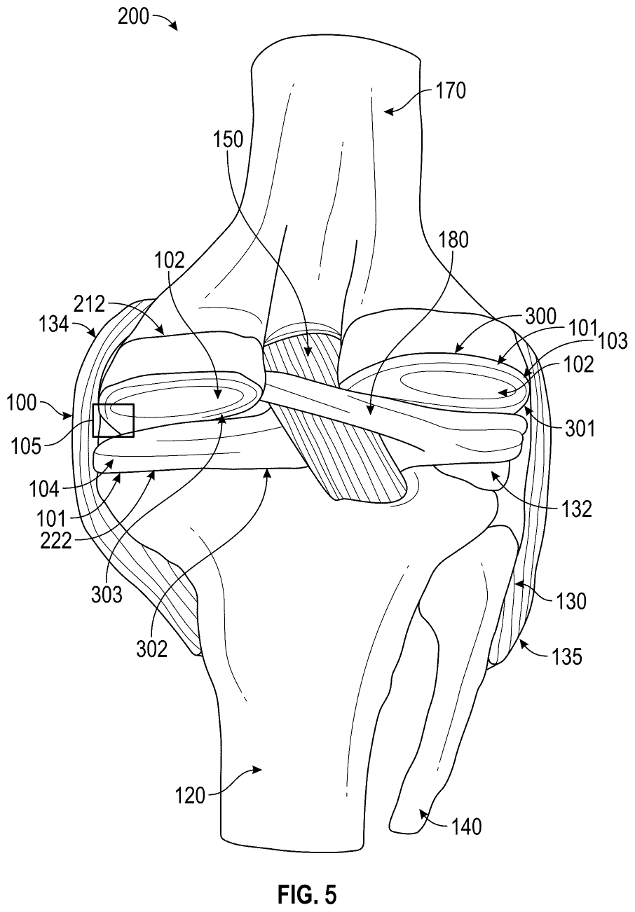

Inverting the placement of porous condyle 102 and insert 104 such that porous condyle 102 is placed on articular surface 222 and insert 104 is placed on opposing surface 212 would lead to rapid degradation of the joint, the development of compromised lower lumbar segments, and, ultimately, the development of potentially chronic lower lumbar pain. After implanting porous condyle 102, first surface 303 of insert 104 and first surface 301 of porous condyle 102 are in a contact relationship with one another to efficiently allow limb joint 200 to articulate between flexed (FIG. 3) and extended (FIGS. 4 and 5) positions as the joint is utilized during physical activity and during a resting position.

In various embodiments, a sensor 150 may be disposed along first surface 303 of insert 104. The sensor 150 may be adhered to first surface 303 using any of the biocompatible adhesives discussed above. The sensor 150 may be configured to measure internal data including temperature, pressure, and/or degradation, and monitor the status of joint replacement system 100 as a result of frequent use and articulation. For example, the sensor 150 may include electro-chemical sensors, impedance sensors, acoustic sensors, among others. The use of the sensor 150 provides the added benefit of enabling external monitoring of the lifespan and/or status of joint replacement system 100, thereby mitigating the need to utilize invasive measures and procedures to observe the joint replacement system 100 when not yet necessary. The sensor 150 may be configured to communicate through a network with a third-party device (e.g., a personal computer, cellular device, or tablet). The sensor 150 may communicate and/or initiate an alert to a user (e.g., the user having joint replacement system 100) regarding health notifications, such as lifespan and/or status issues. While shown on insert 104, it is contemplated that sensor 150 may be placed anywhere on the joint replacement system 100, e.g., on a portion of porous condyle 102.

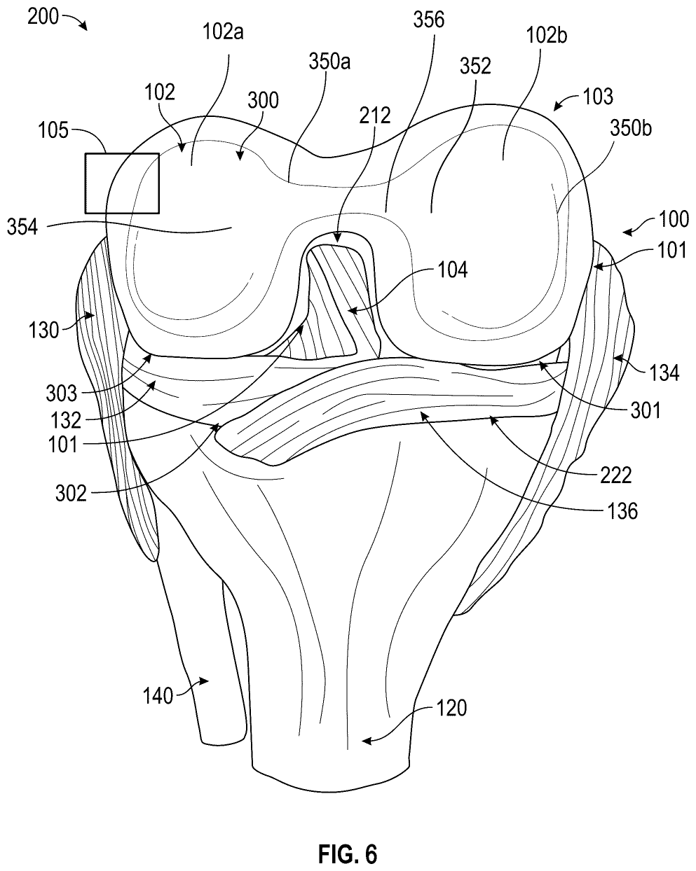

Referring to FIGS. 4 and 6, a diagram of a lateral to medial and anterior non-flexion view of the joint replacement system 100 of FIG. 3 is shown. In the extended position, first surface 301 of porous condyle 102 is in direct contact with first surface 303 of insert 104. The pressure placed upon first surface 301 of porous condyle 102 while in the extended position is distributed across first surface 303 of insert 104 such that a patient will not experience joint discomfort, the degradation of the joint will be significantly decelerated, and the likelihood of lower lumbar pain development will be minimized. The positioning of first surface 301 of porous condyle 102 on the surface directly opposing first surface 303 of insert 104 promotes efficient limb joint 200 articulation and, ultimately, reduces the likelihood of harmful friction occurring within the limb joint 200.

Referring to FIG. 6, a diagram of an anterior flexion view of porous condyle 102, which is filled with biocompatible fluid 103 and positioned upon an articular surface 222 of limb joint 200, is shown. FIG. 6 depicts the physical relationship between first surface 301 of porous condyle 102 and first surface 303 of insert 104. First surface 303 of insert 104 is configured to absorb any shock or pressure that is placed upon first surface 301 of porous condyle 102 when a patient moves and/or otherwise utilizes their joint. Porous condyle 102 includes an exterior surface 350a defining a recess 352 configured to separate a first curvilinear portion 102a of the porous condyle 102 from a second curvilinear portion 102b of the porous condyle 102, the recess 352 disposed between the first 102a and second curvilinear portions 102b of the porous condyle 102. Porous condyle 102 further includes an interior surface 350b defining a cavity 354 configured for containing a biocompatible fluid 103 and a connector member 356 disposed within the cavity 354 between the first 120a and second curvilinear portions 102b of the porous condyle 102. Connector member 356 is configured to enable fluid communication within the cavity 354 between the first 102a and second curvilinear portions 102b of the porous condyle 102.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein. Specific structural and functional details disclosed herein are not to be interpreted as limiting, but as a basis for the claims and as a representative basis for teaching one skilled in the art to variously employ the present disclosure in virtually any appropriately detailed. Like reference numerals may refer to similar or identical elements throughout the description of the figures.

The phrases “in an aspect,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A joint replacement system comprising:
an insert configured to be disposed on an articular surface of a limb joint, the insert fabricated at least in part from polytetrafluoroethylene;
a biocompatible fluid including a synthetic synovial fluid;
a porous condyle configured to contain the biocompatible fluid and disposed on an opposing surface of the limb joint, the porous condyle fabricated at least in part from a rubberized biocompatible material, wherein the porous condyle includes:
an exterior surface defining a recess configured to separate a first curvilinear portion of the porous condyle from a second curvilinear portion of the porous condyle, the recess disposed between the first and second curvilinear portions of the porous condyle;
an interior surface defining a cavity for containing the biocompatible fluid and a diffusible sac containing an additional volume of biocompatible fluid;
a channel disposed along the exterior surface, the channel configured to drain of excess biocompatible fluid within the porous condyle; and
a connector member disposed within the cavity between the first and second curvilinear portions of the porous condyle, the connector member configured to enable fluid communication within the cavity between the first and second curvilinear portions of the porous condyle,
wherein the porous condyle and the insert are aligned to enable articulation of the limb joint,
wherein a first surface of the insert is continuous and configured to contact the porous condyle and a second opposing surface of the insert is configured to be disposed on the articular surface of the limb joint, and
wherein the insert is secured to the articular surface of the limb joint by a biocompatible adhesive including at least one of an ultraviolet (UV) curable adhesive or a biocompatible adhesive spray; and
a sensor disposed along a first surface of the insert.

2. The joint replacement system of claim 1, wherein the limb joint is a knee joint.

3. The joint replacement system of claim 1, wherein the porous condyle is configured to selectively receive an amount of the biocompatible fluid therein.

4. The joint replacement system of claim 1, wherein the biocompatible fluid further includes at least one of glucosamine and a biomimetic synovial fluid.

5. The joint replacement system of claim 1, further comprising a sponge disposed along the opposing surface of the limb joint upon which the porous condyle rests.

6. The joint replacement system of claim 1, wherein the diffusible sac is configured to selectively release an amount of the biocompatible fluid when a decrease of an existing volume of biocompatible fluid within the cavity of the porous condyle is detected.

7. The joint replacement system of claim 1, further comprising a plurality of channels.

8. The joint replacement system of claim 1, wherein the channel is configured to selectively lubricate the porous condyle.

9. The joint replacement system of claim 1, wherein the insert and the porous condyle are configured to be in a contact relationship with one another when the limb joint is in both a flexed and extended position.

10. The joint replacement system of claim 1, wherein during articulation of the limb joint, a surface of the porous condyle is configured to distribute a pressure applied thereon across a surface of the insert, the surface of the insert configured to absorb the applied pressure.

11. The joint replacement system of claim 1, wherein the sensor is configured to detect pressure application across a surface of the insert in response to articulation of the limb joint.

12. The joint replacement system of claim 1, wherein the sensor is configured to detect degradation of the porous condyle in response to articulation of the limb joint.

13. A joint replacement system comprising:
an insert configured to be disposed on an articular surface of a limb joint, the insert fabricated at least in part from polytetrafluoroethylene;
a biocompatible fluid including a synthetic synovial fluid; and
a porous condyle configured for being disposed on an opposing surface of a limb joint, the porous condyle comprising:
a first exterior surface made at least in part from a rubberized biocompatible material defining a recess configured to separate a first curvilinear portion of the porous condyle from a second curvilinear portion of the porous condyle, the recess disposed between the first and second curvilinear portions of the porous condyle;
a second exterior surface encasing the first exterior surface, wherein the second exterior surface is coated in a biocompatible coating including calcium phosphate;
an interior surface defining a cavity for containing a biocompatible fluid including a synthetic synovial fluid;
a diffusible sac within the cavity, wherein the diffusible sac is configured to contain an additional volume of biocompatible fluid;
a channel disposed along the second exterior surface, wherein the channel is configured for drainage of excess biocompatible fluid within the porous condyle; and
a connector member disposed within the cavity between the first and second curvilinear portions of the porous condyle, the connector member configured to enable fluid communication within the cavity between the first and second curvilinear portions of the porous condyle.

14. The joint replacement system of claim 13, wherein the limb joint is a knee joint.

15. The joint replacement system of claim 13, wherein the biocompatible fluid further includes at least one of glucosamine and a biomimetic synovial fluid.

16. The joint replacement system of claim 13, further comprising a sponge disposed along the opposing surface of the limb joint upon which the porous condyle rests.

17. The joint replacement system of claim 13, wherein the channel is further configured to selectively lubricate the porous condyle.

18. The joint replacement system of claim 13, further comprising a sensor disposed along a first surface of the insert.

19. The joint replacement system of claim 18, wherein the sensor is configured to detect at least one of pressure application across a surface of the insert in response to articulation of the limb joint and degradation of the porous condyle in response to articulation of the limb joint.

20. A joint replacement system comprising:
an insert configured to be disposed on an articular surface of a limb joint, the insert fabricated at least in part from polytetrafluoroethylene;
a biocompatible fluid including a synthetic synovial fluid;
a porous condyle configured to contain the biocompatible fluid and disposed on an opposing surface of the limb joint, the porous condyle fabricated at least in part from a rubberized biocompatible material, wherein the porous condyle includes:
a first exterior surface made at least in part from a rubberized biocompatible material defining a recess configured to separate a first curvilinear portion of the porous condyle from a second curvilinear portion of the porous condyle, the recess disposed between the first and second curvilinear portions of the porous condyle;
a second exterior surface encasing the first exterior surface, wherein the second exterior surface is coated in a biocompatible coating including calcium phosphate;
an interior surface defining a cavity for containing a biocompatible fluid including a synthetic synovial fluid;
a diffusible sac within the cavity, wherein the diffusible sac is configured to contain an additional volume of biocompatible fluid;
a channel disposed along the second exterior surface, wherein the channel is configured for drainage of excess biocompatible fluid within the porous condyle; and
a connector member disposed within the cavity between the first and second curvilinear portions of the porous condyle, the connector member configured to enable fluid communication within the cavity between the first and second curvilinear portions of the porous condyle,
wherein the porous condyle and the insert are aligned to enable articulation of the limb joint,
wherein a first surface of the insert is continuous and configured to contact the porous condyle and a second opposing surface of the insert is configured to be disposed on the articular surface of the limb joint, and
wherein the insert is secured to the articular surface of the limb joint by a biocompatible adhesive including at least one of an ultraviolet (UV) curable adhesive or a biocompatible adhesive spray; and
a sensor disposed along a first surface of the insert.
